(19) United States
(12) Patent Application Publication
(10) Pub. No.: US 2025/0005374 A1
(43) Pub. Date: Jan. 2, 2025

(54) INFORMATION PROCESSING DEVICE AND INFORMATION PROCESSING METHOD

(71) Applicants: Preferred Networks, Inc., Tokyo-to (JP); ENEOS Corporation, Tokyo (JP)

(72) Inventors: So TAKAMOTO, Tokyo-to (JP); Chikashi SHINAGAWA, Tokyo-to (JP); Takafumi ISHII, Tokyo (JP)

(73) Assignees: Preferred Networks, Inc., Tokyo-to (JP); ENEOS Corporation, Tokyo (JP)

(21) Appl. No.: 18/884,988

(22) Filed: Sep. 13, 2024

Related U.S. Application Data
(63) Continuation of application No. PCT/JP2023/010158, filed on Mar. 15, 2023.

(30) Foreign Application Priority Data
Mar. 15, 2022 (JP) 2022-040762

(51) Int. Cl. G06N 3/096; G06N 3/04

(52) U.S. Cl. CPC G06N 3/096; G06N 3/04

(57) ABSTRACT

An information processing device includes one or more memories and one or more processors. The one or more processors are configured to input information regarding an atom of a substance to a first model; and obtain information regarding the substance from the first model. The first model is a model which includes: layers from an input layer up to a predetermined layer of a second model to which information regarding atoms is input and which outputs at least one of a value of an energy or a value of a force; and another layer, and which is trained to output the information regarding the substance.

### CROSS REFERENCE TO THE RELATED APPLICATION

This application is continuation application of International Application No. JP2023/010158, filed on Mar. 15, 2023, which claims priority to Japanese Application No. 2022-040762, filed on Mar. 15, 2022, the entire contents of which are incorporated herein by reference.

### FIELD

This disclosure relates to an information processing device and an information processing method.

### BACKGROUND

In the atomic simulation field, a Neural Network Potential (NNP) which is a neural network model trained based on data obtained through quantum chemical calculation or the like is now beginning to be utilized for finding a force field (energy, force).

### BRIEF DESCRIPTION OF THE DRAWINGS

FIG. 1 is a diagram illustrating an example of a model forming a NNP according to an embodiment.

FIG. 2 to FIG. 14 are diagrams illustrating examples of the configuration of a neural network model according to the embodiment.

FIG. 15 and FIG. 16 are flowcharts illustrating processes by an information processing device according to the embodiment.

FIG. 17 is a diagram illustrating an example of the implementation of the information processing device according to the embodiment.

### DETAILED DESCRIPTION

According to one embodiment, an information processing device includes one or more memories and one or more processors. The one or more processors are configured to input information regarding an atom of a substance to a first model; and obtain information regarding the substance from the first model. The first model is a model which includes: layers from an input layer up to a predetermined layer of a second model to which information regarding atoms is input and which outputs at least one of a value of an energy or at a value of a force; and another layer, and which is trained to output the information regarding the substance.

An embodiment of the present invention will be hereinafter described with reference to the drawings. The drawings and the description of the embodiment are presented by way of example only and are not intended to limit the present invention.

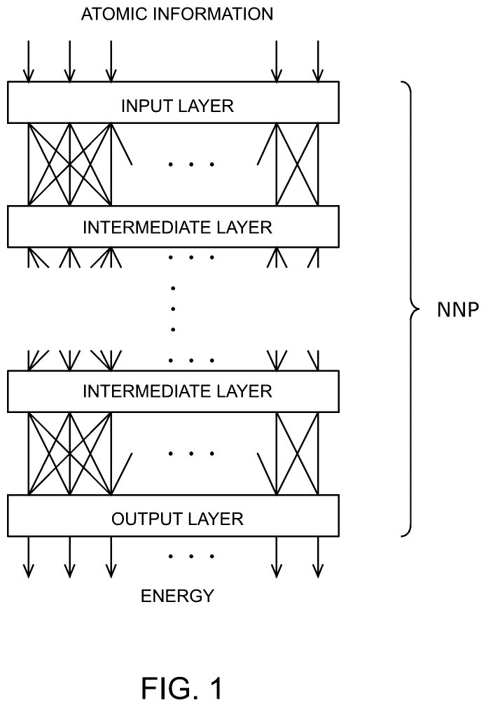

FIG. 1 is a diagram illustrating an example of the configuration of a network model of a Neural Network Potential (NNP) according to the embodiment. The model forming the NNP may be configured using, for example, a Multi-Layer Perceptron (MLP). This MLP may be, for example, a graph neural network to/from which a graph is inputtable/outputtable. Intermediate layers of the following models each may be configured to function as a partial layer of the graph neural network, that is, may be configured such that graph information is inputtable/outputtable thereto/therefrom.

The model forming the NNP illustrated in FIG. 1 is, for example, a model trained using, as training data, an interatomic interaction (energy) obtained through quantum chemical calculation. The model forming the NNP outputs an energy from its output layer when information regarding atoms forming a substance is input to its input layer.

In this embodiment, for example, nodes of the input layer of the model forming the NNP correspond to atoms forming a substance, and information regarding the atoms of the substance is received node by node. Similarly, the output layer of the model forming the NNP outputs the energy in the input state using the node-by-node information. Backpropagating this energy also makes it possible to obtain a force received by each atom.

Information on atoms input to the model forming the NNP is, for example, information including information on the types and positions of the atoms. In this specification, information on atoms will be sometimes called information regarding atoms. Examples of the information on the positions of atoms include information directly indicating the positions of the atoms by means of coordinates and information directly or indirectly indicating the relative position between atoms. This information is expressed by interatomic distance, angle, dihedral angle, and so on.

For example, by calculating pieces of information on the distance between two atoms and the angle among three atoms from information on the coordinates of the atoms and inputting these as information on the positions of the atoms to the model forming the NNP, it is possible to ensure the invariance to rotation/translation to enhance the accuracy of the NNP. For example, the information on atoms may be information directly indicating the positions or may be information calculated from the positional information. Further, the information on atoms may include information regarding electric charges and information regarding bonding besides the information on the types and positions of the atoms.

In a neural network model, typically, input information is gradually updated to output-target information in each layer. Therefore, an output from a given intermediate layer in the model forming the NNP can be considered as a quantity having a feature connecting the information on the atoms and the information on the energy.

In this embodiment, a neural network model capable of inferring the property of a substance using outputs from intermediate layers in the model forming the NNP is formed.

A processing circuit of an information processing device that executes the training of the model may change the output from the output layer of the model forming NNP illustrated in FIG. 1 to a different feature quantity when executing the training. When executing the training, the processing circuit uses a model pre-trained as a model forming a NNP or newly forms a neural network model forming a NNP.

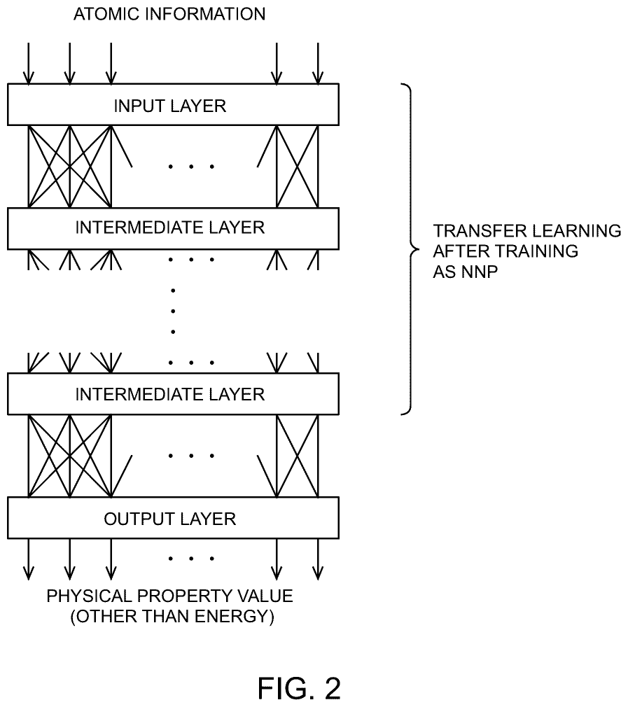

FIG. 2 is a diagram illustrating a nonlimiting example of the formation of the model according to this embodiment. The information processing device of this embodiment obtains various physical property values using a model having layers up to a predetermined intermediate layer of the model (hereinafter, referred to as a second model) forming the NNP illustrated in FIG. 1. The model used may be a model (hereinafter, referred to as a first model) that outputs a physical property value different from the information (energy, force) that can be obtained through the NNP. This disclosure mainly describes the configuration and so on of this first model.

Layers of the second model typically have nodes in the number of atoms. That is, the input layer up to the output layer of the second model each have the same number of nodes as the number of the atoms. Therefore, it can be assumed that as an output from any intermediate layer, some feature quantity corresponding to each atom is also output. In this disclosure, a network that uses the outputs from the intermediate layers of the second model to output another property is connected, and the training is further executed, thereby obtaining a model that infers the other property.

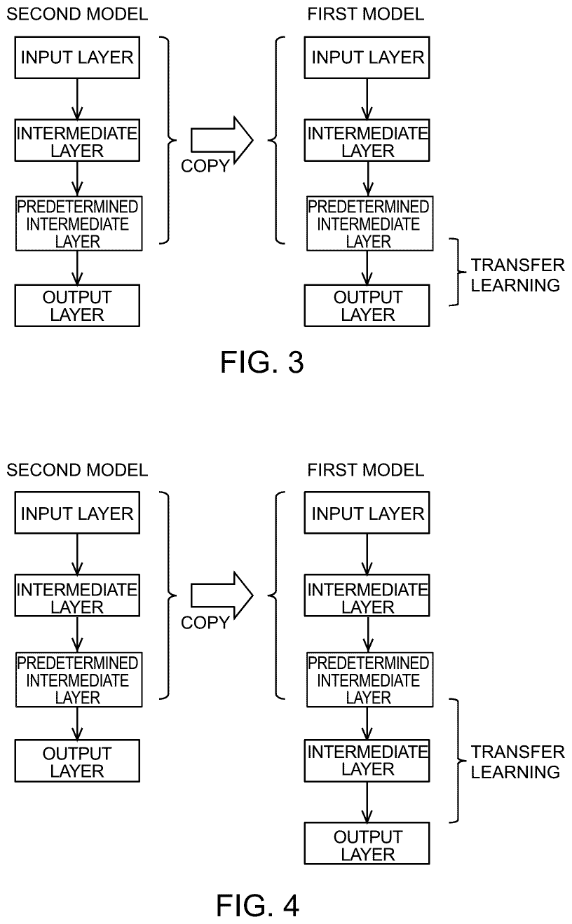

FIG. 3 is a diagram schematically illustrating an example of the generation of the first model in FIG. 2 from FIG. 1. As illustrated in FIG. 3, the first model includes: the input layer up to a predetermined intermediate layer copied from the second model; and a newly connected output layer, thereby capable of outputting information different from that output from the second model. In this case, by training (transfer learning) parameters indicating the connection between the predetermined intermediate layer and the output layer using appropriate training data, it is possible to generate a neural network model that obtains a desired physical property value. Note that in FIG. 3, the predetermined intermediate layer is a layer immediately preceding the output layer of the second model.

The configuration in FIG. 3 is a configuration with two intermediate layers, but this is not limiting. For example, the second model may have a plurality of layers preceding the predetermined intermediate layer, or its layer next to the input layer may be the predetermined intermediate layer. In the first model, parameters of the input layer up to the predetermined intermediate layer of the second model can be appropriately copied and optimized (trained) by transfer learning.

It should be noted that the output layer of the first model is not limited to that additionally connected to the predetermined intermediate layer of the second model, and the predetermined intermediate layer of the second model may be the output layer of the first model.

In this embodiment, the first model and the second model output different pieces of information, that is, the first model outputs information other than an energy or a force, but this is not limiting. As another nonlimiting example, the first model may output the same kind of information as that output from the second model. It is expected that the use of the first model allows the processing circuit, for example, to obtain the same physical property value (substantially the same physical property value) as or a similar physical property value to that obtained from the second model by using a model whose calculation cost is lower than that taken for the outputting from the output layer of the second model.

Further, the first model and the second model may output different kinds of energies or forces. For example, in the case where the model forming the NNP, which is the second model, outputs “a total energy”, the first model may infer a physical property value such as, for example, an adsorption energy or an activation energy.

FIG. 4 is a diagram illustrating another example of the generation of the first model. As illustrated in this diagram, the first model further includes an intermediate layer between the predetermined intermediate layer and the output layer. The first model may thus have a different second intermediate layer instead of the configuration where information is directly propagated from the predetermined intermediate layer to the output layer. This makes it possible to execute a more complicated arithmetic operation on a feature quantity output from the predetermined intermediate layer, and for some inference target, there is a possibility that a better result can be obtained than that obtained in FIG. 3.

In the first model illustrated in FIG. 4, there is one intermediate layer between the predetermined intermediate layer and the output layer of the first model, but this is not limiting. The first model may include a plurality of intermediate layers between the predetermined intermediate layer and the output layer.

FIG. 5 is a diagram illustrating another example of the generation of the first model. As illustrated in this diagram, the first model may be configured such that information is directly propagated also from the intermediate layer other than the predetermined intermediate layer. In this case as well, parameters of the input layer up to the predetermined intermediate layer of the first model may be the same as the parameters of the input layer up to the predetermined intermediate layer of the second model.

Further, the information directly propagated may be one not from the intermediate layer. For example, information may be propagated directly from the input layer to the output layer.

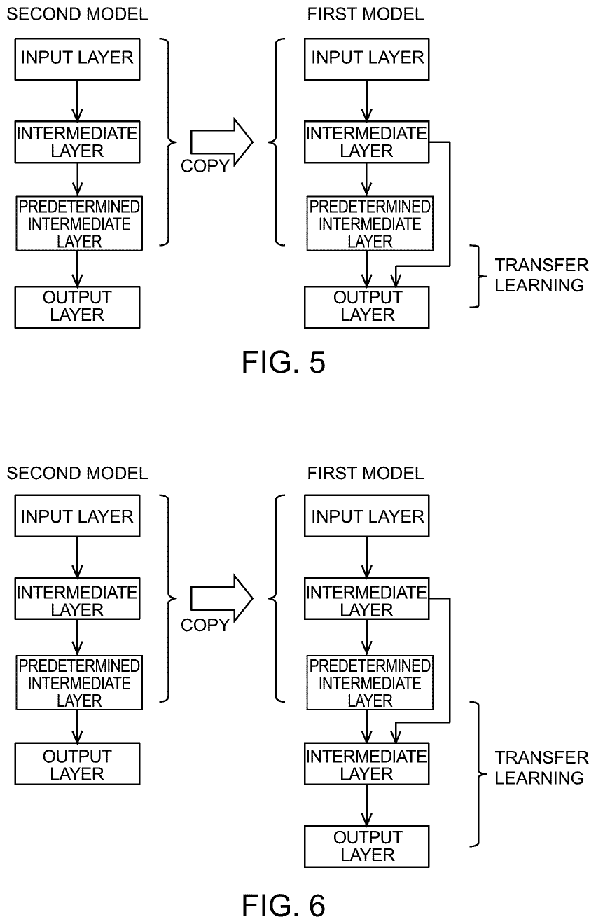

FIG. 6 is a diagram illustrating another example of the generation of the first model. As illustrated in this diagram, the first model may have one or more intermediate layers between the predetermined intermediate layer and the output layer. Information may be propagated directly from the intermediate layer other than the predetermined intermediate layer to the intermediate layer posterior to the predetermined intermediate layer. Further, similarly to the above, information may be propagated directly to the intermediate layer posterior to the predetermined intermediate layer from the input layer instead of from the intermediate layer.

In the case where the plurality of intermediate layers is present between the predetermined intermediate layer and the output layer, information may be propagated directly from the intermediate layer preceding the predetermined intermediate layer to at least one of the intermediate layers posterior to the predetermined intermediate layer. Further, information may be propagated directly from the predetermined intermediate layer to the plurality of intermediate layers posterior to the predetermined intermediate layer. Further, in the configuration where the plurality of intermediate layers posterior to the predetermined intermediate layer are present, information may be directly propagated from the intermediate layer preceding the predetermined intermediate layer to the output layer as in FIG. 5.

Similarly to the above, in the first model, the number of the intermediate layers between the input layer and the predetermined intermediate layer (the intermediate layers preceding the predetermined intermediate layer) and the number of the intermediate layers between the predetermined intermediate layer and the output layer (the intermediate layers posterior to the predetermined intermediate layer) may be any. Therefore, the propagation of information from an intermediate layer to an intermediate layer in FIG. 6 may be propagation of the information from any of the one or more intermediate layers preceding the predetermined intermediate layer to any of the one or more intermediate layers posterior to the predetermined intermediate layer.

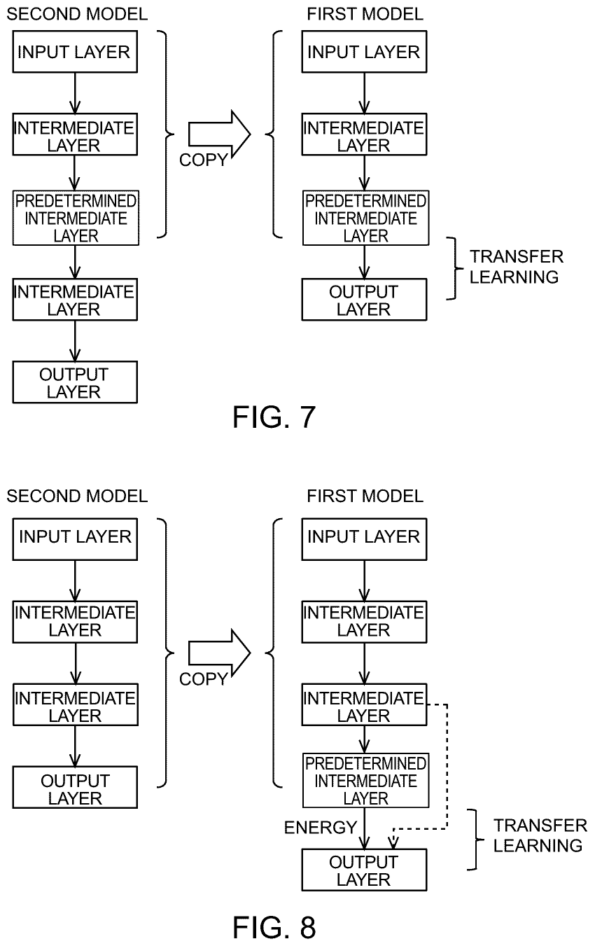

FIG. 7 is a diagram illustrating another example of the generation of the first model. As illustrated in this diagram, the predetermined intermediate layer may be a layer not immediately preceding the output layer in the second model. Further, in this case, the configuration of the first model may be any of the configurations illustrated in FIG. 4 to FIG. 6. That is, the first model may include one or more intermediate layers between the predetermined intermediate layer and the output layer, or may be configured such that information can be directly propagated from the intermediate layer preceding the predetermined intermediate layer to an intermediate layer posterior to the predetermined intermediate layer or to the output layer.

FIG. 8 is a diagram illustrating another example of the generation of the first model. In the first model, the output layer of the second model may be formed as the predetermined intermediate layer. In this case, from the predetermined intermediate layer, the value of the energy is output as in the second model. The parameters between predetermined intermediate layer and the output layer may be trained so that this output is converted to desired information.

Further, as indicated by the dotted-line arrow, information may be propagated from the intermediate layer preceding the predetermined intermediate layer of the first model to the output layer. Further, these examples are not limiting, and an intermediate layer may be arranged after the predetermined intermediate layer as illustrated in FIG. 4, or information may be propagated from the intermediate layer preceding the predetermined intermediate layer directly to the intermediate layer posterior to the predetermined intermediate layer as illustrated in FIG. 6.

In FIG. 3 to FIG. 8, several examples have been presented but they are not limiting. For example, in the layers from the input layer up to the predetermined intermediate layer, a layer for dimensional compression or dimensional expansion may be present.

In the case where the input layer and the output layer each have the same number of nodes as the number of atoms, the predetermined intermediate layer preferably has the same number of nodes as that in the input layer and the output layer because this enables the predetermined intermediate layer to output data of each atom from each node, but this is not limiting. For example, the predetermined intermediate layer may be a layer where the compression of the nodes or the expansion of the nodes (in other words, dimensional compression or dimensional expansion) is done.

Further, although the intermediate layer can be variously arranged, with the parameters of copied parts being fixed, the first model may have connection such that information can be propagated between at least two layers including, the input layer, any intermediate layers, or the output layer.

Further, in the above, the parameters of the input layer up to the predetermined intermediate layer in the first model are the same as the parameters of the input layer up to the predetermined intermediate layer in the second model, but this is not limiting. That is, the first model may be a model that is fine-tuned so as to give a different output using the parameters obtained in the model forming the NNP.

Further, in the first model, a model formed following the predetermined intermediate layer is not limited to a neural network model. For example, a different machine learning model such as a random forest may be connected to the predetermined intermediate layer. Further, the layers and parameters that the first model has are not limited to those of the MLP but may be those of a neural network model of another form.

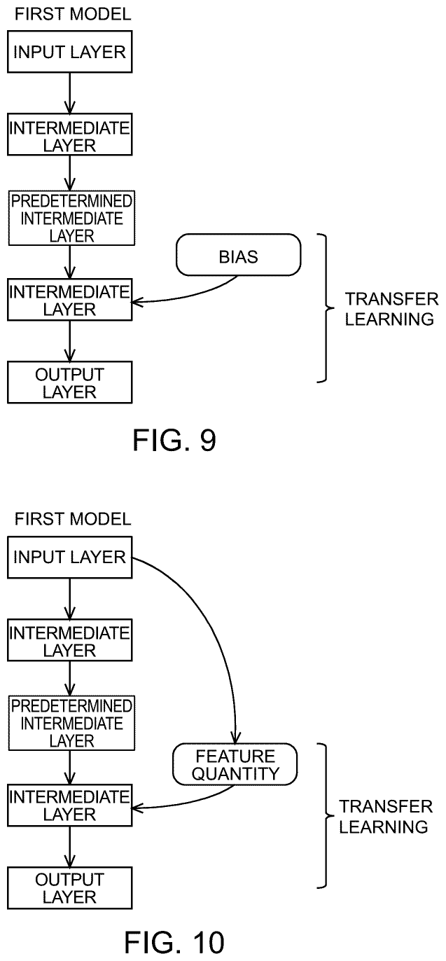

FIG. 9 is a diagram illustrating still another example of the first model. In the configurations of the models in FIG. 3 to FIG. 8, the input of a bias is not shown, but a bias may be appropriately input as illustrated in FIG. 9. For example, the first model may execute inference by applying the input bias to the output of the predetermined intermediate layer.

FIG. 10 is a diagram illustrating still another example of the first model. Another value (feature quantity) for the input atomic composition may be separately obtained by the second model, and the second model may also be optimized to a model, as the first model, having undergone transfer learning or fine-tuning using this separately obtained value and the value of the predetermined intermediate layer. For example, the first model may be a model that executes inference by weight-adding the value of the predetermined intermediate layer to a fingerprint obtained from the input information on atoms.

The feature quantity may be an already defined feature quantity, such as the aforesaid fingerprint, obtained based on a predetermined algorithm. As another example, for the calculation of the feature quantity, another neural network may be formed for the input to the input layer. In this case, the other neural network may also be one that has been trained as part of transfer learning.

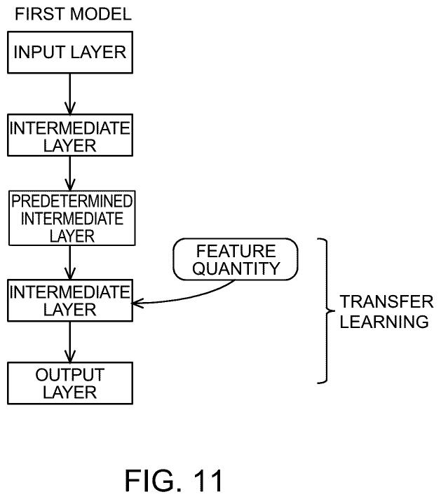

FIG. 11 is a diagram illustrating another example of FIG. 10. The first model may be configured such that a feature quantity that has been separately obtained in the information on atoms input to the input layer of the first model can be input to the intermediate layer posterior to the predetermined intermediate layer of the first model. Another employable form is to separately form a neural network model that obtains the feature quantity from the input information regarding the atoms and input an output of this neural network to the intermediate layer posterior to the predetermined intermediate layer.

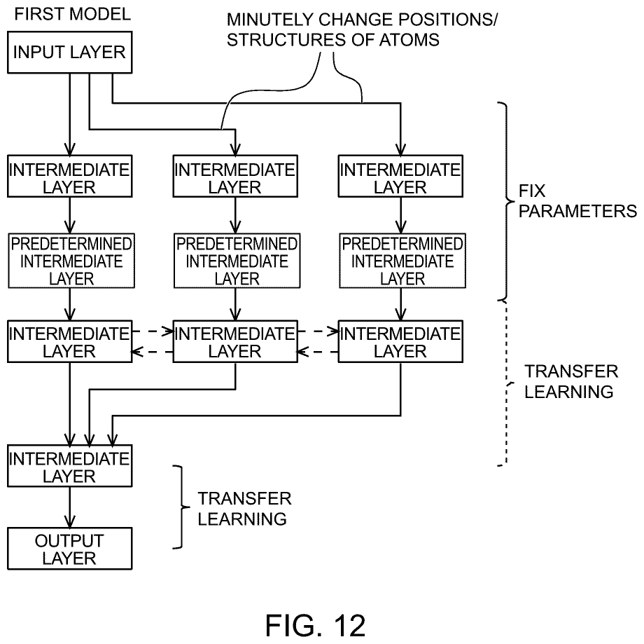

FIG. 12 is a diagram illustrating still another example of the first model. Pieces of information regarding a plurality of chemical structures generated for the input atomic composition are pieces of input information to intermediate layers arranged in parallel. For example, the first model is a model that branches into multiple (in FIG. 12, three) paths, which are parallelized, using the parameters of the input layer up to the predetermined intermediate layer which parameters are obtained from the second model, and on a subsequent stage, integrates data output from the respective branches to output the integrated data.

Note that the broken-line arrows in FIG. 12 represent weighting parameters that can be set as desired. For example, the first model may be configured to propagate outputs to/from intermediate layers following the predetermined intermediate layers in the parallel network structures. The broken-line arrows are not indispensable, and the connections indicated by the broken-line arrows do not necessarily have to be present in the first model.

In the case without the broken-line arrows, in the first model, data are propagated in parallel in the intermediate layers relevant to the plurality of chemical structures. The broken-line arrows illustrated in FIG. 12 indicate that information obtained from a certain chemical structure is input to an intermediate layer that processes another chemical structure. In this case, the connection of the intermediate layers relevant to the plurality of chemical structures includes a series connection. For example, in the case where the plurality of chemical structures corresponds to structural changes accompanying a chemical reaction, exchanging the pieces of information on the chemical structures as illustrated in this first model is advantageous for inferring the easiness of the chemical reaction.

Note that even in the case where the connections indicated by the broken-line arrows are present, the propagations indicated by all the broken-line arrows need not be implemented simultaneously in the first model. It is not excluded that the first model in FIG. 12 has at least one data input/output indicated by the broken-line arrow out of those indicated by the broken-line arrows in the parallel propagation paths.

Note that parallel and series mentioned here are as follows. In FIG. 12, connections indicated by the solid-line arrows across INPUT LAYER→INTERMEDIATE LAYER→PREDETERMINED INTERMEDIATE LAYER→INTERMEDIATE LAYER→INTERMEDIATE LAYER in the left array and across the same structure in the middle array or the right array are called parallel propagation paths. On the other hand, the broken-line arrow from the left intermediate layer to the middle intermediate layer or the broken-line arrow from the middle intermediate layer to the left intermediate layer is called a series connection. The same applies to the middle path and the right path. Further, the left path and the right path may be connected in series.

The branching from the input layer to the parallel paths includes, for example, branching where the information input to the input layer is output as it is to the intermediate layers and branching where it is output after being given one or more minute changes. The minute change may be a change corresponding to a minute change in the position, structure, or the like of an atom in a graph, for instance.

In the layers up to the predetermined intermediate layers in the first model, the parameters obtained from the second model may be fixed for use.

On a subsequent stage of the predetermined intermediate layers in the respective branches, pieces of information output from the parallel models in the first model are output from the output layer through an intermediate layer that integrates the outputs. In the respective paths in the branches of the first model, intermediate layers for adjusting the respective outputs may be further provided between the predetermined intermediate layers and the intermediate layer that integrates the outputs, as illustrated in FIG. 12.

The parameters relevant to the intermediate layer and the output layer at and after the integration processing are tuned by transfer learning or the like previously explained in the description up to FIG. 11. Further, as indicated by the dotted-line parenthesis, in the case where the intermediate layers are further present between the predetermined intermediate layers and the intermediate layer that integrates the outputs of the minutely changed structures, parameters relevant to the intermediate layers preceding the integration may be tuned.

Examples of the plurality of chemical structures and predicted information are as follows, but those in the embodiment in this disclosure are not limited to these.

For example, information regarding the plurality of chemical structures resulting from the minute distance displacement of part or all of the atoms in the original structure may be given as an input. Based on this input, a differential value (for example, a Hessian matrix) regarding the atomic nucleus coordinates of the original structure may be obtained. Further, thermodynamic quantities (for example, an enthalpy at a given temperature) that can be calculated from this differential value can be predicted.

Further, two different chemical structures may be given as the input. From this input, it is also possible to predict an X parameter which is a parameter indicating inter-structural anti-affinity between these two chemical structures. In the case where the X parameter is predicted, the two chemical structures each may be a molecule or may be a constituent element of a polymer. Further, for the higher-precision prediction of the X-parameter, the volume of one of the two chemical structures is preferably not less than 0.125 nor more than 8 times the volume of the other, and an average volume of these is preferably 1 nm³ or less.

Note that, though the first model has the three parallel paths in FIG. 12 and the above description, the number of the parallel paths is not limited to this and may be two or may be four or more.

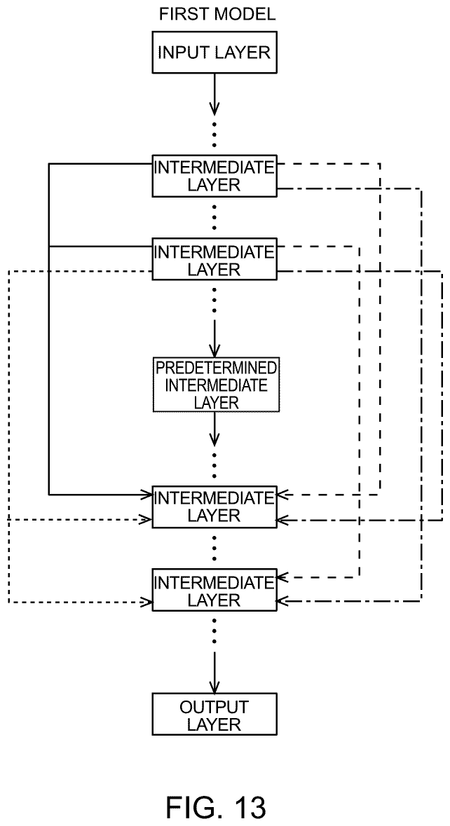

FIG. 13 is a diagram illustrating several nonlimiting examples of a variation of the aforesaid output from the intermediate layer between the input layer and the predetermined intermediate layer to the intermediate layer between the predetermined intermediate layer and the output layer.

As indicated by the left solid-line arrows, outputs may be given from a plurality of intermediate layers to one intermediate layer.

As indicated by the dotted-line arrows, an output may be given from one intermediate layer to a plurality of intermediate layers.

As indicated by the broken-line or dash-dot-line arrows, outputs may be given from a plurality of intermediate layers to a plurality of different intermediate layers.

These are presented only by way of example, and the connection between the intermediate layers can be in any form as described above. For example, the intermediate layers between the input layer and the predetermined intermediate layer, which are not in a connection relationship in the second model, may be connected so that information can be propagated directly between these, or the intermediate layers between the predetermined intermediate layer and the output layer may be connected through a more complicated network configuration to enable the propagation of information between these through this network.

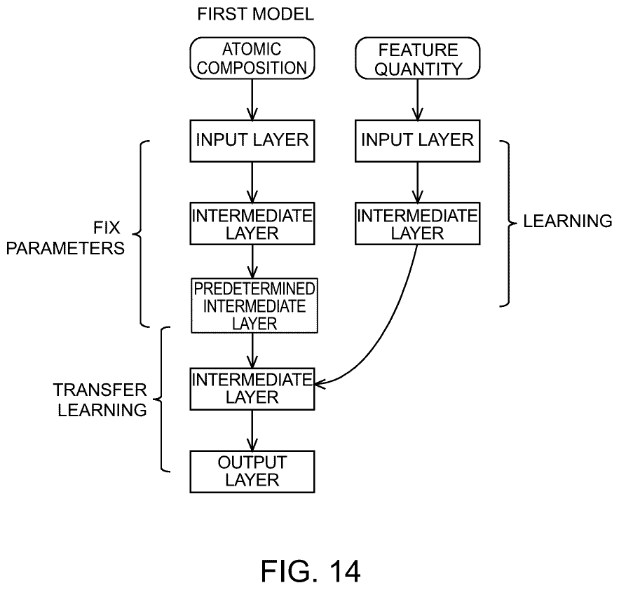

FIG. 14 is a diagram illustrating still another example of the first model. This first model separately obtains another value (feature quantity) that cannot be obtained from the input atomic composition, and this is input to an input layer different from and parallel to the input layer to which the atomic composition is input. A network continuing from the input layer corresponding to the feature quantity may have one or more intermediate layers different from those of the second model.

As in the above-described modes, in the first model, the input layer up to the predetermined intermediate layer corresponding to the atomic composition may have the fixed parameters of the second model.

In the first model, information output from the intermediate layer following the parallel input layer corresponding to the feature quantity may be propagated to a given layer posterior to the layers from the input layer up to the predetermined intermediate layer corresponding to the atomic composition. The first model can output information other than an energy and a force from the output layer after integrating the information obtained from the atomic composition and the information obtained from the feature quantity.

Nonlimiting several examples of the feature quantity other than the atomic composition include information on temperature, pressure, time, fraction, and so on. Training data for information that is desired to be obtained in the case where these nonlimiting feature quantities are input is prepared, and parameters relevant to the layers in the parts indicated by transfer learning and learning in FIG. 14 are optimized. As a result, it is possible to form a trained model that is capable of inferring a physical property value or the like desired to be obtained for various kinds of environment information that can be converted to numerical values.

By enabling the inputting of the feature quantity other than the atomic composition to the input layer, it is possible to form the first model that predicts viscosity, a reaction rate constant, and so on which are nonlimiting examples of the information other than the energy and the force.

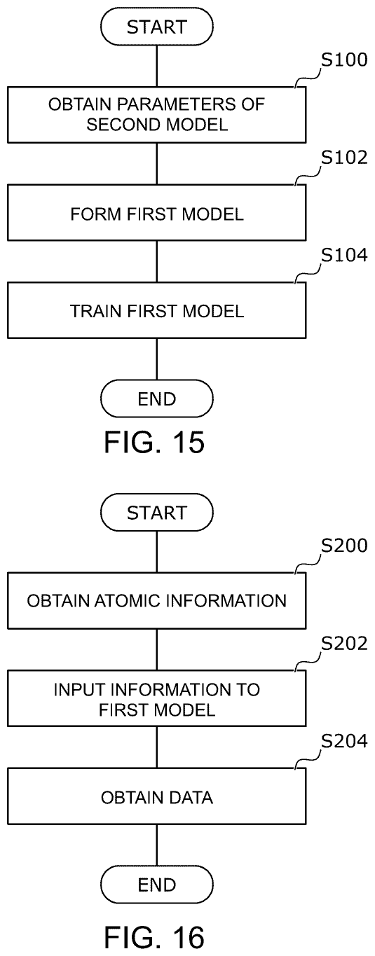

FIG. 15 is a flowchart illustrating a process where the information processing device according to the embodiment trains the first model.

The processing circuit of the information processing device first obtains parameters of the second model (S100). The parameters may be obtained from a pre-trained model or may be those trained by the same information processing device. The processing circuit obtains, in particular, information regarding layers and interlayer information used to configure the first model, out of the configuration of the second model.

Next, based on the parameters obtained from the second model, the processing circuit forms the first model (S102). The processing circuit copies information such as the parameters to places, in the first model, common to the second model, and appropriately arranges additional layers to form the configuration of the first model.

Next, the processing circuit trains the first model (S104). The processing circuit trains the first model by, for example, transfer learning. For the training, the processing circuit uses, as training data, data of atoms forming a substance and information that is desired to be obtained such as a physical property value or the like in the data of the atoms.

After the training is appropriately finished, the parameters and so on are output, and the process is ended.

FIG. 16 is a flowchart illustrating a process of inferring by the information processing device according to the embodiment using the first model.

The processing circuit of the information processing device first obtains atomic information in a substance whose value is desired to be obtained (S200). This atomic data may be graph information.

The processing circuit inputs the obtained atomic data to the first model (S202). The processing circuit forward propagates the data which is input through the input layer, thereby inferring and obtaining desired data (S204). A desired quantity can be thus inferred using the first model.

As described above, according to this embodiment, the transfer learning using the model forming the NNP makes it possible to obtain various pieces of other highly accurate information regarding atoms and a substance.

The intermediate layer of the model forming the NNP outputs, for each atom, values which are multidimensional quantities (for example, 100 values for each atom, or the like). These quantities are expected to have information expressing an ambient environment-based state (for example, a bonding state, an oxidation number, and so on) of each atom, depending on the function of the neural network.

Further, the NNP has characteristics that physical simulation-based data can be used as training data and a model excellent in generalization performance can be easily generated. Therefore, it can be expected that the use of such a model for inferring other information makes it possible to obtain a highly accurate result. Further, by the predetermined intermediate layer having the same number of nodes as the number of nodes of the input layer and the output layer, it is possible to obtain a feature quantity for each atom or each bond forming the substance. As a result, it is possible to appropriately use the feature quantity of each atom to obtain another value.

An energy that can be obtained from the model forming the NNP has a clear physical definition. Therefore, high-precision calculation, for example, the calculation of a theoretical value is possible. In the case where atoms, molecules, and the like are handled, it is usually necessary to define a quantity of electric charges or the like, but such a quantity is difficult to clearly define. Further, an energy has an extensive property and can be superposed, or the like. Therefore, it can be expected that, in an intermediate layer close to the output layer of the model forming the NNP, for example, in the immediately preceding intermediate layer in the second model, each node appropriately contains information regarding the relevant atom. Therefore, according to the model of this disclosure, it can be expected that it is possible to appropriately obtain various data and so on regarding objects or atoms by using the output from such an intermediate layer. Note that the information contained in the intermediate layer sometimes has substance-related information not linked with a bond or a specific atom, besides the information on each atom.

The output of the first model may be, for example, various physical property values, optical properties, mechanical properties, an influence on an organism, or the like of a molecule, an environment, and so on. As a typical example, the first model may be formed as a model that outputs a Highest Occupied Molecular Orbital (HOMO) energy, a Lowest Unoccupied Molecular Orbital (LUMO) energy, an X parameter, or a fingerprint. As a result, it is also possible to infer the solubility or pH of a substance. As another example, the first model may be formed as a model that performs clustering or visualization. As a result, it can be considered using this as an index of whether or not a certain molecule belongs to a crystal, whether or not it is similar to a crystal, or the like. Further, the first model may be configured to output the information regarding the substance from its layer other than the output layer.

The X parameter expresses a nondimensionalized energy in the case where two atomic groups are in contact with each other, and a known method for its calculation is a method based on the Monte-Carlo method, molecular dynamics, or the like, but they require high calculation costs. It can be expected that the use of the first model formed in this disclosure can reduce the calculation cost.

Note that, in the embodiment described above, the output layer of the model (second model) forming the NNP may be configured to output at least one of an energy of a system, an energy of an atom, and a force applied to an atom.

The trained model in the above-described embodiment may be, for example, a concept further including a model distilled by a typical method after it is trained in the above-described manner.

Further, a model generation method of training and generating the first model using the above-described information processing device is naturally included in the scope of this disclosure.

To summarize the above description, in this disclosure, the expression that
“the first model includes:
   layers from an input layer up to a predetermined intermediate layer of a second model; and
   another layer,”
      implies at least one of the following two concepts.
<1>The first model is a model that is formed using (1) the layers from the input layer up to the predetermined intermediate layer (predetermined layer) of the second model and (2) the other layer, and thereafter is trained by transfer learning where the values of (1) are fixed.
<2>The first model is a model that is formed using (1) the layers from the input layer up to the predetermined intermediate layer (predetermined layer) of the second model and (2) the other layer, and thereafter is trained by fine tuning that updates the values of (1) and (2) by learning. This includes a case where the values of (1) are at least partly updated. For example, a case where the values of the layers from the input layer up to a certain intermediate layer of the second model are fixed and the other parameters in the second model are updated is included.

The trained models of above embodiments may be, for example, a concept that includes a model that has been trained as described and then distilled by a general method.

Some or all of each device (the information processing device) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

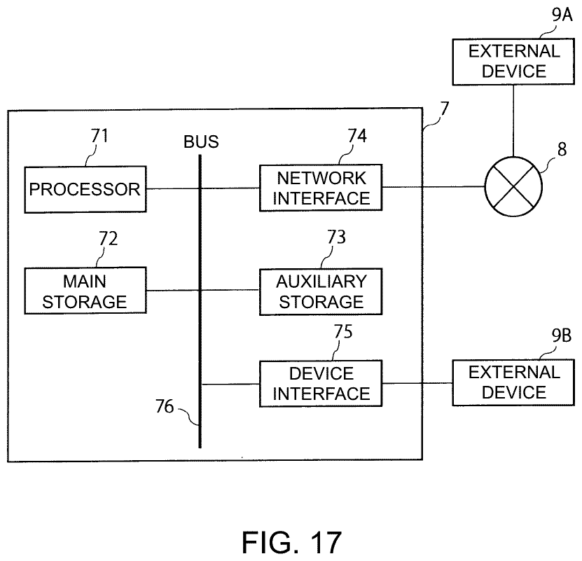

FIG. 17 is a block diagram illustrating an example of a hardware configuration of each device (the information processing device) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 17 is provided with each component one by one but may be provided with a plurality of the same components. Although one computer 7 is illustrated in FIG. 17, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the information processing device) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the information processing device) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuity, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the information processing device) in the above embodiments may be enabled by one or more processors 71. The processor 71 may refer to one or more electronic circuits located on one chip, or one or more electronic circuitries arranged on two or more chips or devices. In the case of a plurality of electronic circuitries is used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the information processing device) in the above embodiments may be enabled by the main storage device 72 or the auxiliary storage device 73 or may be implemented by a built-in memory built into the processor 71. For example, the storages in the above embodiments may be implemented in the main storage device 72 or the auxiliary storage device 73.

In the case of each device (the information processing device) in the above embodiments is configured by at least one storage device (memory) and at least one processor connected/coupled to/with this at least one storage device, the at least one processor may be connected to a single storage device. Or the at least one storage may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the information processing device) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrical connection/coupling,” “communicative connection/coupling,” “operative connection/coupling,” “physical connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize/minimization” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize/optimization” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing device comprising:
one or more memories; and
one or more processors configured to:
input information regarding atoms of a substance to a first model; and
obtain information regarding the substance from the first model,
wherein the first model is a model
which includes: layers from an input layer up to a predetermined layer of a second model to which information regarding atoms is input and which outputs at least one of a value of an energy or a value of a force; and another layer, and
which is trained to output the information regarding the substance.

2. The information processing device according to claim 1,
wherein the first model is a model which is trained by transfer learning using the layers from the input layer up to the predetermined layer of the second model.

3. The information processing device according to claim 1,
wherein the first model is a model which is fine-tuned using the layers from the input layer up to the predetermined layer of the second model.

4. The information processing device according to claim 1,
wherein the first model outputs the information regarding the substance using at least one of an output of the input layer or one or more outputs of one or more layers different from the predetermined layer in the second model.

5. The information processing device according to claim 1,
wherein the first model is a model in which the predetermined layer of the second model and an output layer of the first model are connected.

6. The information processing device according to claim 1,
wherein the first model includes one or more intermediate layers between the predetermined layer of the second model and the output layer of the first model.

7. The information processing device according to claim 1,
wherein the predetermined layer is an intermediate layer of the second model.

8. The information processing device according to claim 7,
wherein the predetermined layer is a layer immediately preceding an output layer of the second model.

9. The information processing device according to claim 1,
wherein the information regarding the substance is a physical property value of the substance.

10. The information processing device according to claim 1,
wherein, for pieces of information regarding a plurality of chemical structures, the first model includes at least one of parallel propagation paths or parallel propagation paths among which at least one series connection of intermediate layers is present, and
wherein the pieces of information to the parallel propagation paths are input from the input layer as the information regarding the atoms of the substance.

11. The information processing device according to claim 1,
wherein the information regarding the atoms of the substance is input in the first model through a layer being the input layer and corresponding to the input layer of the second model, and in parallel to the input layer, the first model includes a different input layer to which a feature quantity other than an atomic composition is input, and
wherein the first model integrates information obtained from the information regarding the atoms of the substance and information obtained from the feature quantity other than the atomic composition and outputs the resultant.

12. The information processing device according to claim 9,
wherein the physical property value of the substance is at least one of a HOMO (Highest Occupied Molecular Orbital) energy, a LUMO (Lowest Unoccupied Molecular Orbital) energy, or an X parameter.

13. The information processing device according to claim 1,
wherein the information regarding the substance is information used for clustering or visualizing the substance.

14. An information processing device comprising:
one or more memories; and
one or more processors configured to:
train a first model to make the first model output information regarding a substance when information regarding atoms of the substance is input,
wherein the first model includes: layers from an input layer up to a predetermined layer of a second model which is a trained model; and another layer, and
wherein the second model is a model which outputs at least one of a value of an energy or a value of a force when information regarding atoms is input.

15. The information processing device according to claim 14,
wherein the one or more processors train the first model by transfer learning using the layers from the input layer up to the predetermined layer of the second model.

16. The information processing device according to claim 14,
wherein the one or more processors train the first model by fine tuning using the layers from the input layer up to the predetermined layer of the second model.

17. The information processing device according to claim 14,
wherein the information regarding the substance is a physical property value of the substance.

18. The information processing device according to claim 17,
wherein the physical property value of the substance is at least one of a HOMO (Highest Occupied Molecular Orbital) energy, a LUMO (Lowest Unoccupied Molecular Orbital) energy, an X parameter, or a fingerprint.

19. The information processing device according to claim 14,
wherein the information regarding the substance is information used for clustering or visualizing the substance.

20. An information processing method comprising:
by one or more processors,
inputting information regarding atoms of a substance to a first model; and
obtaining information regarding the substance from the first model,
wherein the first model is a model
which includes layers from an input layer up to a predetermined layer of a second model to which information regarding atoms is input and which outputs at least one of a value of an energy or a value of a force, and
which is trained to output information regarding the substance.
